(19) United States
(12) Patent Application Publication
(10) Pub. No.: US 2024/0108558 A1
(43) Pub. Date: Apr. 4, 2024

(54) COMPOSITION FOR CARING FOR THE SKIN

(71) Applicant: L'OREAL, Paris (FR)

(72) Inventor: Yuanji GUO, Shanghai (CN)

(73) Assignee: L'OREAL, Paris (FR)

(21) Appl. No.: 18/257,022

(22) PCT Filed: Dec. 16, 2020
(86) PCT No.: PCT/CN2020/136820

§ 371 Date: Jun. 12, 2023

(51) Int. Cl. A61K 8/37; A61K 8/31; A61K 8/92; A61Q 17/00

(52) U.S. Cl. CPC A61K 8/37; A61K 8/31; A61K 8/922; A61Q 17/00; A61K 2800/31; A61K 2800/48

(57) ABSTRACT

It relates to an anhydrous composition for caring for the skin, in particular the scalp, comprising: (i) at least one linear alkane containing from 10 to 28 carbon atoms; (ii) at least one oil of formula R₁COOR₂ in which R₁ represents a linear or branched fatty acid residue containing from 4 to 40 carbon atoms and R₂ represents a hydrocarbon-based chain containing from 4 to 40 carbon atoms, on condition that R₁+R₂≥16; and (iii) at least one vegetable oils of triglyceride type. It also relates to a non-therapeutic method for caring for the skin, in particular the scalp, comprising applying the composition to the skin, in particular the scalp.

### TECHNICAL FIELD

The present invention relates to a composition. In particular, the present invention relates to a composition for caring for the skin, in particular the scalp. The present invention also relates to a non-therapeutic process for caring for the skin, in particular the scalp.

### BACKGROUND ART

The skin constitutes a physical barrier between the body and its surroundings. It is constituted of two tissues: the epidermis and the dermis.

The dermis provides the epidermis with a solid support. It is also its nourishing element. It is mainly constituted of fibroblasts and an extracellular matrix, which is itself composed mainly of collagen, elastin and a substance known as ground substance, these components being synthesized by the fibroblast. Leukocytes, mast cells or else tissue macrophages are also found therein. It also contains blood vessels and nerve fibres.

The epidermis is a desquamating pluristratified epithelium that is 100 μm thick on average and is conventionally divided into a basal layer of keratinocytes that constitutes the germinal layer of the epidermis, a spinous layer constituted of several layers of polyhedral cells positioned on the germinal cells, a granular layer constituted of flattened cells containing distinct cytoplasmic inclusions, keratohyalin granules, and finally an upper layer known as the cornified layer (or stratum corneum), constituted of keratinocytes at the terminal stage of their differentiation, known as corneocytes. These are mummified anucleate cells which derive from keratinocytes. The stack of these corneocytes constitutes the cornified layer that is responsible, inter alia, for the barrier function of the epidermis, i.e. it constitutes a barrier against external attacks, especially chemical, mechanical or infectious attacks and it also makes it possible to protect the body from water loss.

In order to change the appearance of the hair, there are many products developed to treat the hair during dyeing, bleaching, waving and/or straightening.

During the application of products for dyeing, bleaching, waving and/or straightening the hair, some ingredients will contact the scalp and pass across the stratum corneum to the living epidermis, which may cause discomfort and irritation to the scalp.

Thus, there is a need to formulate a cosmetic composition for caring for the skin, in particular the scalp, which can prevent water-soluble dyes and strongly alkaline components from passing across the stratum corneum to the living epidermis.

### SUMMARY OF THE INVENTION

An object of the present invention is to provide a composition for caring for the skin, in particular the scalp, which can prevent water-soluble dyes and strongly alkaline components from passing across the stratum corneum to the living epidermis.

Another object of the present invention is to provide a composition for caring for the skin, in particular the scalp, which will not adversely impact the dyeing and/or bleaching effect of a dyeing and/or bleaching product on the hair.

Accordingly, in a first aspect, the present invention relates to an anhydrous composition for caring for the skin, in particular the scalp, comprising:
(i) at least one linear alkane containing from 10 to 28 carbon atoms;
(ii) at least one oil of formula R₁COOR₂ in which R₁ represents a linear or branched fatty acid residue containing from 4 to 40 carbon atoms and R₂ represents a hydrocarbon-based chain containing from 4 to 40 carbon atoms, on condition that R₁+R₂≥16; and
(iii) at least one vegetable oils of triglyceride type.

The composition of the present invention can be in the form of oil gel or oil balm.

In a second aspect, the present invention relates to a non-therapeutic method for caring for the skin, in particular the scalp, comprising applying the composition according to the first aspect of the present invention to the skin, in particular the scalp.

It was found that the composition according to the present invention can prevent water-soluble dyes and strongly alkaline components from passing across the stratum corneum to the living epidermis and avoid the scalp being stained by the dyes. Meanwhile, the composition according to the present invention has no negative impact on the dyeing effect of a dyeing composition or the bleaching effect of a bleaching product on the hair.

Other subjects and characteristics, aspects and advantages of the present invention will emerge even more clearly on reading the detailed description and the examples that follow.

### BRIEF DESCRIPTION OF THE DRAWINGS

Implementations of the present invention will now be described, by way of example only, with reference to the attached figures, wherein:

FIG. 1 shows the fluorescence intensity in stratum corneum upon application of a dyeing composition, wherein 1 (the pink moiety in FIG. 1) stands for water soluble fluorescent dye, 2 (the blue moiety in FIG. 1) stands for keratinocyte and keratin, and 3 (the green moiety in FIG. 1) stands for collagen.

FIG. 2 shows the fluorescence intensity in stratum corneum upon application of the composition of comparative formula 1 and a dyeing composition, wherein 1 (the pink moiety in FIG. 2) stands for water soluble fluorescent dye, 2 (the blue moiety in FIG. 2) stands for keratinocyte and keratin, and 3 (the green moiety in FIG. 2) stands for collagen.

FIG. 3 shows the fluorescence intensity in stratum corneum upon application of the composition of invention formula 2 and a dyeing composition, wherein 2 (the blue moiety in FIG. 3) stands for keratinocyte and keratin, and 3 (the green moiety in FIG. 3) stands for collagen.

FIG. 4 shows the fluorescence intensity in stratum corneum upon application of the composition of invention formula 3 and a dyeing composition, wherein 1 (the pink moiety in FIG. 4) stands for water soluble fluorescent dye, 2 (the blue moiety in FIG. 4) stands for keratinocyte and keratin, and 3 (the green moiety in FIG. 4) stands for collagen.

FIG. 5 shows the amount of formazan for different treatments, wherein “Ngc” stands for the original cell viability, “alone” stands for the cell viability without protection, “shawarzkopf” stands for the cell viability under the protection of a product from Schwarzkopf, N5 stands for the cell viability under the protection of the composition of invention formula 3, N8 stands for the cell viability under the protection of the composition of invention formula 2, and N9 stands for the cell viability under the protection of the composition of invention formula 1.

FIG. 6 shows photos taken during evaluation by staining on a pig skin.

FIG. 7 shows photos taken during evaluation with hair swatches and model's heads.

FIG. 8 shows photos taken during evaluation with hair swatches.

### DETAILED DESCRIPTION OF THE INVENTION

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

All percentages in the present invention refer to weight percentage, unless otherwise specified.

For the purposes of the present invention, the term “anhydrous” means that the composition according to the present invention contains less than 2 wt. % and preferably less than 0.5 wt. % of water relative to the total weight of the composition. Where appropriate, such small amounts of water may be provided by ingredients of the composition that contain it in residual amount but are not deliberately provided. In particular, there is no water in the composition.

According to the first aspect, the present invention provides an anhydrous composition for caring for the skin, in particular the scalp, comprising:
(i) at least one linear alkane containing from 10 to 28 carbon atoms;
(ii) at least one oil of formula R₁COOR₂ in which R₁ represents a linear or branched fatty acid residue containing from 4 to 40 carbon atoms and R₂ represents a hydrocarbon-based chain containing from 4 to 40 carbon atoms, on condition that R₁+R₂≥16; and
(iii) at least one vegetable oils of triglyceride type.

Linear Alkanes

According to the first aspect, the composition according to the present invention comprises at least one linear alkane containing from 10 to 28 carbon atoms.

As examples of linear alkanes suitable for use in the composition of the present invention, mention can be made of n-decane (C10), n-undecane (C11), n-dodecane (C12), n-tridecane (C13), n-tetradecane (C14), n-pentadecane (C15), n-hexadecanol (C16), n-heptadecane (C17), n-octacosane (C18), n-nonadecane (C19), n-eicosane (C20), n-heneicosane (C21), n-docosane (C22), n-tricosane (C23), n-tetracosane (C24), n-pentacosane (C25), n-hexacosane (C26), n-heptacosane (C27), n-octacosane (C28), and mixtures thereof.

Preferably, the linear alkance is selected from linear C12-C24 alkane.

More preferably, the linear alkane is selected from linear C14-C22 alkane.

In some embodiments, the linear alkane used is linear C15-C19 alkane.

As commercial product of linear alkanes, mention can be made of C15-19 alkane sold under the name EMOGREEN™ L19 by the company SEPPIC.

Advantageously, the linear alkane is present in an amount ranging from 0.1 wt. % to 10 wt. %, preferably from 0.2 wt. % to 5 wt. %, more preferably from 0.5 wt. % to 3 wt. %, relative to the total weight of the composition.

Oils of Formula R₁COOR₂

According to the first aspect, the composition according to the present invention comprises at least one oil of formula R₁COOR₂.

In formula R₁COOR₂, R₁ represents a linear or branched fatty acid residue containing from 4 to 40 carbon atoms and R₂ represents a hydrocarbon-based chain containing from 4 to 40 carbon atoms, on condition that R₁+R₂≥16.

As examples of oil of formula R₁COOR₂, mention can be made of purcellin oil (cetostearyl octanoate), isononyl isononanoate, 2-ethylhexyl palmitate, octyldodecyl neopentanoate, stearyl heptanoate, stearyl caprylate, 2-octyldodecyl stearate, 2-octyldodecyl erucate, oleyl erucate, isostearyl isostearate, 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate or 2-diethylhexyl succinate.

Preferably, the oil of formula R₁COOR₂ is selected from esters of a C6-C24 monocarboxylic acid and of a C4-C20 alcohol, at least one of the acid and the alcohol being branched or unsaturated; more preferably selected from esters of a C12-C20 monocarboxylic acid and of a C4-C10 alcohol, at least one of the acid and the alcohol being branched or unsaturated.

Most particularly the oil of formula R₁COOR₂ is selected from:
C4-C10 alkyl myristates, especially isobutyl myristate, isohexyl myristate, 2-ethylhexyl myristate or isodecanyl myristate;
C4-C10 alkyl palmitates, especially isobutyl palmitate, isohexyl palmitate, 2-ethylhexyl palmitate or isodecanyl palmitate;
C4-C10 alkyl isostearates, especially isobutyl isostearate, isohexyl isostearate, 2-ethylhexyl isostearate or isodecanyl isostearate, and
mixtures thereof.

As commercial product of oil of formula R₁COOR₂, mention can be made of 2-ethylhexyl palmitate sold under the name CEGESOFT® C 24 by the company BASF.

Advantageously, the oil of formula R₁COOR₂ is present in an amount ranging from 0.1 wt. % to 10 wt. %, preferably from 0.2 wt. % to 5 wt. %, more preferably from 0.5 wt. % to 3 wt. %, relative to the total weight of the composition.

Vegetable Oils of Triglyceride Type

According to the first aspect, the composition according to the present invention comprises at least one vegetable oil of triglyceride type.

Preferably, the vegetable oils of triglyceride type are selected from sunflower seed oil, corn oil, soybean oil, marrow oil, grapeseed oil, sesame seed oil, hazelnut oil, apricot oil, macadamia oil, castor oil, olive oil; avocado oil; argan oil; camellia oil, palm oil, maize germ oil, cottonseed oil, peanut oil, pumpkin seed oil, wheatgerm oil, babassu oil, coconut oil, rapeseed oil, almond oil, linseed oil, safflower oil, and also mixtures thereof.

More preferably, the vegetable oils of triglyceride type are selected sunflower seed oil, jojoba seed oil, Argania spinosa kernel oil, coconut oil, corn oil, soybean oil, marrow oil, grapeseed oil, sesame seed oil, hazelnut oil, apricot oil, macadamia oil, castor oil, and avocado oil.

As commercial product of vegetable oil of triglyceride type, mention can be made of Helianthus annuus (sunflower) seed oil sold under the name REFINED SUNFLOWER OIL by the company AAK KAMANI PRIVATE, SIMMONDSIA CHINENSIS (JOJOBA) SEED OIL sold under the name JD FFL JOJOBA GOLDEN OIL by the company JOJOBA DESERT, Cocos nucifera (coconut) oil sold under the name ACTIVATED VIRGIN COCONUT OIL by the company BIOTROPICS, ARGANIA SPINOSA KERNEL OIL sold under the name LIPOFRUCTYL ARGAN BE LS 9779 by the company BASF BEAUTY CARE SOLUTION, and Prunus armeniaca (apricot) kernel oil sold under the name APRICOT KERNEL OIL REFINED DAC by the company GUSTAV HEESS.

Advantageously, the vegetable oil of triglyceride type is present in an amount ranging from 1 wt. % to 30 wt. %, preferably from 3 wt. % to 20 wt. %, more preferably from 8 wt. % to 15 wt. %, relative to the total weight of the composition.

Semi-Crystalline or Crystalline Lipophilic Thickener

Preferably, the composition according to the present invention comprises a semi-crystalline or crystalline lipophilic thickener.

The term “lipophilic” here means a substance, which is soluble in oil(s) at a concentration of at least 1 wt. % relative to the total weight of the oil(s) at room temperature (25° C.) and atmosphere pressure (10⁵ Pa).

The semi-crystalline or crystalline lipophilic thickener used in the present invention can be selected from semi-crystalline or crystalline polymers.

The semi-crystalline or crystalline polymer is preferably a semi-crystalline polymer. The term “semi-crystalline polymer” means polymers comprising a crystallizable portion, a crystallizable pendent and/or end chain or a crystallizable block in the backbone and/or at the ends, and an amorphous portion in the backbone, and having a first-order reversible temperature of change of phase, in particular of melting (solid-liquid transition). When the crystallizable portion is in the form of a crystallizable block of the polymer backbone, the amorphous portion of the polymer is in the form of an amorphous block; the semi-crystalline polymer is, in this case, a block copolymer, for example of the diblock, triblock or multiblock type, comprising at least one crystallizable block and at least one amorphous block. The term “block” generally means at least five identical repeating units. The crystallizable block(s) are then of different chemical nature from the amorphous block(s).

The semi-crystalline polymer according to the present invention has a melting point of greater than or equal to 30° C. (especially ranging from 30° C. to 80° C.), preferably ranging from 30° C. to 60° C., and in particular ranging from 40° C. to 50° C. This melting point is a first-order temperature of change of state.

This melting point may be measured by any known method and in particular using a differential scanning calorimeter (DSC), for example the calorimeter sold under the name DSC Q2000 by the company TA Instruments.

Advantageously, the semi-crystalline polymer(s) to which the present invention applies has a number-average molecular mass of greater than or equal to 1000.

Advantageously, the semi-crystalline polymer(s) of the composition of the invention has a number-average molecular mass ranging from 2000 to 800 000, preferably from 3000 to 500 000, better still from 4000 to 150 000 and especially less than 100 000 and better still from 4000 to 99 000. Preferably, they have a number-average molecular mass of greater than 5600, for example ranging from 5700 to 99 000.

For the purposes of the present invention, the expression “crystallizable chain or block” means a chain or block which, if it were obtained alone, would change from the amorphous state to the crystalline state reversibly, depending on whether the temperature is above or below the melting point. For the purposes of the present invention, a “chain” is a group of atoms, which are pendent or lateral relative to the polymer backbone. A “block” is a group of atoms belonging to the backbone, this group constituting one of the repeating units of the polymer. Advantageously, the “pendent crystallizable chain” may be a chain containing at least 6 carbon atoms.

Preferably, the crystallizable block(s) or chain(s) of the semi-crystalline polymers represent at least 30% of the total weight of each polymer and better still at least 40%. The semi-crystalline polymers of the present invention containing crystallizable blocks are block or multiblock polymers. They may be obtained via polymerization of a monomer containing reactive double bonds (or ethylenic bonds) or via polycondensation. When the polymers of the present invention are polymers containing crystallizable side chains, these side chains are advantageously in random or statistical form.

Preferably, the semi-crystalline polymers that may be used in the composition according to the present invention are of synthetic origin. Moreover, they do not comprise a polysaccharide backbone. In general, the crystallizable units (chains or blocks) of the semi-crystalline polymers according to the invention originate from monomer(s) containing crystallizable block(s) or chain(s), used for the manufacture of the semi-crystalline polymers.

According to the invention, the semi-crystalline polymer may be chosen from block copolymers comprising at least one crystallizable block and at least one amorphous block, and homopolymers and copolymers bearing at least one crystallizable side chain per repeating unit, and mixtures thereof.

The semi-crystalline polymers that may be used in the invention are in particular:
block copolymers of polyolefins with controlled crystallization, especially those whose monomers are described in EP-A-0 951 897,
polycondensates, especially of aliphatic or aromatic polyester type or of aliphatic/aromatic copolyester type,
homopolymers or copolymers bearing at least one crystallizable side chain and homopolymers or copolymers bearing at least one crystallizable block in the backbone, for instance those described in document U.S. Pat. No. 5,156,911,
homopolymers or copolymers bearing at least one crystallizable side chain, in particular containing fluoro group(s), as described in document WO-A-01/19333,
and mixtures thereof.

In the last two cases, the crystallizable side chain(s) or block(s) are hydrophobic.

(i) Semi-Crystalline Polymers Containing Crystallizable Side Chains

Mention may be made in particular of those defined in documents U.S. Pat. No. 5,156,911 and WO-A-01/19333. They are homopolymers or copolymers comprising from 50% to 100% by weight of units resulting from the polymerization of one or more monomers bearing a crystallizable hydrophobic side chain.

These homopolymers or copolymers are of any nature, provided that they meet the conditions mentioned previously.

They can result:
from the polymerization, especially the free-radical polymerization, of one or more monomers containing reactive or ethylenic double bond(s) with respect to a polymerization, namely a vinyl, (meth)acrylic or allylic group,
from the polycondensation of one or more monomers bearing co-reactive groups (carboxylic acid, sulfonic acid, alcohol, amine or isocyanate), such as, for example, polyesters, polyurethanes, polyethers, polyureas or polyamides.

In general, these polymers are chosen especially from homopolymers and copolymers resulting from the polymerization of at least one monomer containing crystallizable chain(s) that may be represented by formula (I):

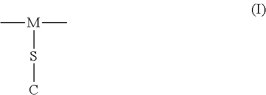

with M representing an atom of the polymer backbone, S representing a spacer and C representing a crystallizable group.

The crystallizable chains “—S—C” may be aliphatic or aromatic, and optionally fluorinated or perfluorinated. “S” especially represents the group (CH₂)ₙ or (CH₂CH₂O)ₙ or (CH₂O), which may be linear or branched or cyclic, with n being an integer ranging from 0 to 22. Preferably, “S” is a linear group. Preferably, “S” and “C” are different.

When the crystallizable chains “—S—C” are hydrocarbon-based aliphatic chains, they comprise hydrocarbon-based alkyl chains containing at least 11 carbon atoms and not more than 40 carbon atoms and better still not more than 24 carbon atoms. They are especially aliphatic chains or alkyl chains containing at least 12 carbon atoms, and they are preferably C14-C24 alkyl chains. When they are fluoroalkyl or perfluoroalkyl chains, they contain at least six fluorinated carbon atoms and especially at least 11 carbon atoms, at least six of which carbon atoms are fluorinated.

As examples of semi-crystalline polymers or copolymers bearing crystallizable chain(s), mention may be made of those resulting from the polymerization of one or more of the following monomers: (meth)acrylates of saturated alkyl with the alkyl group being C14-C24, perfluoroalkyl (meth)acrylates with a C11-C15 perfluoroalkyl group, N-alkyl(meth)acrylamides with the alkyl group being C14 to C24 with or without a fluorine atom, vinyl esters containing alkyl or perfluoro(alkyl) chains with the alkyl group being C14 to C24 (with at least 6 fluorine atoms per perfluoroalkyl chain), vinyl ethers containing alkyl or perfluoro(alkyl) chains with the alkyl group being C14 to C24 and at least 6 fluorine atoms per perfluoroalkyl chain, C14 to C24 alpha-olefins such as, for example, octadecene, para-alkylstyrenes with an alkyl group containing from 12 to 24 carbon atoms, and mixtures thereof.

When the polymers result from a polycondensation, the hydrocarbon-based and/or fluorinated crystallizable chains as defined above are borne by a monomer that may be a diacid, a diol, a diamine or a diisocyanate.

When the polymers that are the subject of the present invention are copolymers, they additionally contain from 0 to 50% of groups Y or Z resulting from the copolymerization:
α) of Y which is a polar or non-polar monomer or a mixture of the two:

When Y is a polar monomer, it is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a non-polar monomer, it may be an ester of the linear, branched or cyclic alkyl (meth)acrylate type, a vinyl ester, an alkyl vinyl ether, an α-olefin, styrene or styrene substituted with a C1 to C10 alkyl group, for instance α-methylstyrene.

For the purposes of the present invention, the term “alkyl” means a saturated group especially of C8 to C30, except where otherwise mentioned, and better still of C10 to C30.
β) of Z which is a polar monomer or a mixture of polar monomers. In this case, Z has the same definition as the “polar Y” defined above.

Preferably, the semi-crystalline polymers containing a crystallizable side chain are homopolymers of C8-C30, preferably C10-C30 alkyl (meth)acrylate or C8-C30, preferably C₁₀-C₃₀alkyl(meth)acrylamide, copolymers of these monomers and a hydrophilic monomer preferably of different nature from (meth)acrylic acid, for instance N-vinylpyrrolidone or hydroxyethyl (meth)acrylate, and mixtures thereof.

(ii) Polymers Bearing in the Backbone at Least One Crystallizable Block

These polymers are especially block copolymers consisting of at least two blocks of different chemical nature, one of which is crystallizable.
The block polymers defined in U.S. Pat. No. 5,156,911 may be used;
Block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-tetrahydronaphthalene, dicyclopenta-diene, or mixtures thereof,
with ethylene, propylene, 1-butene, 3-methyl-1-butene, 1-hexene, 4-methyl-1-pentene, 1-octene, 1-decene or 1-eicosene, or mixtures thereof.
and in particular copoly(ethylene/norbornene) blocks and (ethylene/propylene/ethylidene-norbornene) block terpolymers. Those resulting from the block copolymerization of at least two C2-C16, better still C2-C12 and even better still C4-C12 α-olefins such as those mentioned above and in particular block bipolymers of ethylene and of 1-octene may also be used.
The copolymers may be copolymers containing at least one crystallizable block, the copolymer residue being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:
Block that is crystallizable by nature: a) of polyester type, for instance poly(alkylene terephthalate), b) of polyolefin type, for instance polyethylenes or polypropylenes.
Amorphous and lipophilic block, for instance amorphous polyolefins or copoly(olefin)s such as poly(isobutylene), hydrogenated polybutadiene or hydrogenated poly(isoprene).

As examples of such copolymers containing a crystallizable block and a separate amorphous block, mention may be made of:
α) poly(ε-caprolactone)-b-poly(butadiene) block copolymers, preferably used hydrogenated, such as those described in the article “Melting behaviour of poly(ε-caprolactone)-block-polybutadiene copolymers” from S. Nojima, Macromolecules, 32, 3727-3734 (1999),
β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
γ) the poly(ethylene)-b-copoly(ethylene/propylene) block copolymers cited in the articles “Morphology of semicrystalline block copolymers of ethylene-(ethylene-alt-propylene)” by P. Rangarajan et al., Macromolecules, 26, 4640-4645 (1993) and “Polymer aggregates with crystalline cores: the system poly(ethylene)poly(ethylene-propylene)” by P. Richter et al., Macromolecules, 30, 1053-1068 (1997).
δ) the poly(ethylene)-b-poly(ethylethylene) block copolymers mentioned in the general article “Crystallization in block copolymers” by I. W. Hamley, Advances in Polymer Science, vol. 148, 113-137 (1999).

The semicrystalline polymers in the composition of the present invention may or may not be partially crosslinked, provided that the degree of crosslinking does not interfere with their dissolution or dispersion in the liquid fatty phase optionally present in the composition by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer backbone; or to a phase separation between the crystallizable blocks and the amorphous blocks, borne by the polymer.

Preferably, the semi-crystalline polymers of the composition according to the present invention are not crosslinked.

According to one particular embodiment of the invention, the polymer is chosen from copolymers resulting from the polymerization of at least one monomer containing a crystallizable chain chosen from saturated C14 to C24 alkyl (meth)acrylates, C11 to C15 perfluoroalkyl (meth)acrylates, C14 to C24 N-alkyl(meth)-acrylamides with or without a fluorine atom, vinyl esters containing C14 to C24 alkyl or perfluoroalkyl chains, vinyl ethers containing C14 to C24 alkyl or perfluoroalkyl chains, C14 to C24 alpha-olefins, para-alkylstyrenes with an alkyl group containing from 12 to 24 carbon atoms, with at least one optionally fluorinated C1 to C10 monocarboxylic acid ester or amide, which may be represented by the following formula (II):

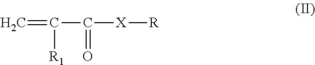

in which R₁ is H or CH₃, R represents an optionally fluorinated C1-C10 alkyl group and X represents O, NH or NR₂ in which R₂ represents an optionally fluorinated C1-C10 alkyl group.

According to one more particular embodiment of the present invention, the polymer is derived from a monomer containing a crystallizable chain chosen from saturated C14 to C22 alkyl (meth)acrylates and even more particularly poly(stearyl acrylate) or poly(behenyl acrylate).

As particular examples of structuring semi-crystalline polymers that may be used in the composition according to the present invention, mention may be made of polymers having the INCI name “Poly C₁₀-C₃₀ alkyl acrylate” (poly(C₁₀-C₃₀ alkyl acrylate)), for instance the Intelimer® products from the company Air Products, for instance the product Intelimer® IPA 13-1, which is a polystearyl acrylate of a melting point of 48° C., or the product Intelimer® IPA 13-6, which is a behenyl polymer.

The semi-crystalline polymers may especially be:
those described in Examples 3, 4, 5, 7, 9 and 13 of U.S. Pat. No. 5,156,911 containing a —COOH group, resulting from the copolymerization of acrylic acid and of C5 to C16 alkyl (meth)acrylate and more particularly of the copolymerization:
   of acrylic acid, of hexadecyl acrylate and of isodecyl acrylate in a 1/16/3 weight ratio,
   of acrylic acid and of pentadecyl acrylate in a 1/19 weight ratio,
   of acrylic acid, of hexadecyl acrylate and of ethyl acrylate in a 2.5/76.5/20 weight ratio,
   of acrylic acid, of hexadecyl acrylate and of methyl acrylate in a 5/85/10 weight ratio,
   of acrylic acid and of octadecyl methacrylate in a 2.5/97.5 weight ratio,
   of hexadecyl acrylate, of polyethylene glycol methacrylate monomethyl ether containing 8 ethylene glycol units, and of acrylic acid in an 8.5/1/0.5 weight ratio.

It is also possible to use the structure “O” from National Starch, as described in document U.S. Pat. No. 5,736,125, with a melting point of 44° C., and also semi-crystalline polymers with crystallizable pendent chains comprising fluoro groups, as described in Examples 1, 4, 6, 7 and 8 of document WO-A-01/19333.

It is also possible to use the semi-crystalline polymers obtained by copolymerization of stearyl acrylate and of acrylic acid or NVP as described in document U.S. Pat. No. 5,519,063 or EP-A-550 745, with melting points of 40° C. and 38° C., respectively.

It is also possible to use the semi-crystalline polymers obtained by copolymerization of behenyl acrylate and of acrylic acid or NVP, as described in documents U.S. Pat. No. 5,519,063 and EP-A-550 745, with melting points of 60° C. and 58° C., respectively.

Preferably, the semi-crystalline polymers do not comprise any carboxylic groups.

Finally, the semi-crystalline polymers according to the present invention may also be chosen from waxy polymers obtained by metallocene catalysis, such as those described in patent application US 2007/0 031 361.

These polymers are homopolymers or copolymers of ethylene and/or propylene prepared via metallocene catalysis, i.e. by polymerization at low pressure and in the presence of a metallocene catalyst.

The weight-average molecular mass (Mw) of the waxes obtained via metallocene catalysis described in that document is less than or equal to 25 000 g/mol and ranges, for example, from 2000 to 22 000 g/mol and better still from 4000 to 20 000 g/mol.

The number-average molecular mass (Mn) of the waxes obtained via metallocene catalysis described in that document is preferably less than or equal to 15 000 g/mol and ranges, for example, from 1000 to 12 000 g/mol and better still from 2000 to 10 000 g/mol.

The polydispersity index I of the polymer is equal to the ratio of the weight-average molecular mass Mw to the number-average molecular mass Mn. Preferably, the polydispersity index of the waxy polymers is between 1.5 and 10, more preferably between 1.5 and 5, even more preferably between 1.5 and 3 and better still between 2 and 2.5.

The waxy homopolymers and copolymers may be obtained in a known manner from ethylene and/or propylene monomers, for example via metallocene catalysis according to the process described in document EP 571 882.

The homopolymers and copolymers of ethylene and/or propylene prepared via metallocene catalysis may be unmodified or “polar”-modified (polar-modified waxes, i.e. waxes modified such that they have the properties of a polar wax). The polar-modified waxy homopolymers and copolymers may be prepared in a known manner from unmodified waxy homopolymers and copolymers such as those described previously by oxidation with gases containing oxygen, such as air, or by grafting with polar monomers such as maleic acid or acrylic acid or alternatively derivatives of these acids. These two routes enabling polar modification of the polyolefins obtained via metallocene catalysis are described, respectively, in documents EP 890 583 and U.S. Pat. No. 5,998,547, for example, the content of these two documents being incorporated herein by reference.

According to the present invention, the polar-modified homopolymers and copolymers of ethylene and/or propylene prepared via metallocene catalysis that are particularly preferred are polymers modified such that they have hydrophilic properties. Examples that may be mentioned include ethylene and/or propylene homopolymers or copolymers modified by the presence of hydrophilic groups such as maleic anhydride, acrylate, methacrylate, polyvinylpyrrolidone (PVP), etc.

Waxy ethylene and/or propylene homopolymers or copolymers modified by the presence of hydrophilic groups such as maleic anhydride or acrylate are particularly preferred.

Examples that may be mentioned include:
polypropylene waxes modified with maleic anhydride (PPMA) sold by the company Clariant, or polypropylene-ethylene-maleic anhydride copolymers, such as those sold by the company Clariant under the name LicoCare, for instance LicoCare PP207 LP3349, LicoCare CM401 LP3345, LicoCare CA301 LP3346 and LicoCare CA302 LP3347,
the unmodified polyethylene waxes sold by the company Clariant, such as the product LicoCare PE 102 LP3329.

Advantageously, the lipophilic thickener is present in an amount ranging from 0.1 wt. % to 10 wt. %, preferably from 0.2 wt. % to 8 wt. %, more preferably from 0.5 wt. % to 5 wt. %, relative to the total weight of the composition.

Additional Oils

The composition according to the present invention may further comprise one or more additional oils besides the oils described above.

The additional oils can be non-volatile oils and/or volatile oils.

The oils may be silicone oil, fluoro oil, hydrocarbon-based oil, or a mixture thereof.

The term “fluoro oil” means an oil comprising at least one fluorine atom.

The oils may optionally comprise oxygen, nitrogen, sulfur and/or phosphorus atoms, for example in the form of hydroxyl or acid radicals.

The silicone oil and fluoro oil may be selected from polar oils, apolar oils, or mixtures thereof.

The “hydrocarbon-based oil” used as the additional oil can be polar oils.

For the purposes of the present invention, the term “polar oil” means an oil solubility parameter thereof at 25° C., δa, is other than 0 (J/cm³)^{1/2}.

According to a preferred embodiment, the composition according to the present invention comprises at least one additional oil, such as squalane.

Advantageously, the total amount of all oils is present in the composition of the present invention ranges from 60 wt. % to 99.5 wt. %, relative to the total weight of the composition.

Additional Adjuvants or Additives

The composition of the present invention may comprise conventional cosmetic adjuvants or additives, for instance fragrances, chelating agents (for example, disodium EDTA), preserving agents (for example, chlorphenesin and phenoxyethanol) and bactericides, fillers, pH regulators (for example citric acid, sodium hydroxide, potassium hydroxide), and mixtures thereof.

The skilled in the art can select the amount of the additional adjuvants or additive so as not to adversely affect the final use of the composition according to the present invention.

According to a particularly preferred embodiment, the present invention provides an anhydrous composition for caring for the skin, in particular the scalp, comprising, relative to the total weight of the composition:
(i) from 0.5 wt. % to 3 wt. % of at least one linear C15-C19 alkane;
(ii) from 0.5 wt. % to 3 wt. % of at least one C4-C10 alkyl palmitate;
(iii) from 8 wt. % to 15 wt. % of at least one vegetable oils of triglyceride type selected sunflower seed oil, jojoba seed oil, Argania spinosa kernel oil, coconut oil, corn oil, soybean oil, marrow oil, grapeseed oil, sesame seed oil, hazelnut oil, apricot oil, macadamia oil, castor oil, and avocado oil; and
(iv) from 0.5 wt. % to 5 wt. % of at least one poly(C10-C30 alkyl acrylate).

Galenic Form and Use

The composition of the present invention can be in the form of oil gel or oil balm.

The viscosity of the composition according to the invention can be measured at 25 C, using a ProRheo R180 viscometer equipped with a spindle M2 or M3 rotating at 200 rpm.

According to a preferred embodiment, the composition of the present invention has a viscosity of from 10 UD (Deviation Units) to 80 UD, preferably from 10 UD to 50 UD, measured at 25° C. using a Rheomat R180 viscometer equipped with a spindle M2 rotating at 200 rpm.

According to the second aspect, the present invention relates to a non-therapeutic method for caring for the skin, in particular the scalp, comprising applying the composition according to the first aspect of the present invention to the skin, in particular the scalp.

The composition according to the present invention can prevent water-soluble dyes and strongly alkaline components from passing across the stratum corneum to the living epidermis.

Meanwhile, the composition according to the present invention has no negative impact on the dyeing effect of a dyeing composition on the hair.

Furthermore, the composition according to the present invention has no negative impact on the bleaching effect of a bleaching product on the hair.

The following examples serve to illustrate the present invention without, however, being limiting in nature.

### EXAMPLES

Main raw materials used, trade names and supplier thereof are listed in Table 1.

TABLE 1

| INCI Name | Trade Name | Supplier |
|---|---|---|
| C15-19 ALKANE | EMOGREEN ™ L19 | SEPPIC |
| SQUALANE | NEOSSANCE ® | AMYRIS |
| | SQUALANE | |
| ETHYLHEXYL | CEGESOFT ® C 24 | BASF |
| PALMITATE | | |
| POLY C10-30 ALKYL | TEGO ® SP 13-1 | EVONIK (AIR |
| ACRYLATE | | PRODUCTS) |
| HELIANTHUS ANNUUS | REFINED | AAK KAMANI |
| (SUNFLOWER) SEED | SUNFLOWER OIL | PRIVATE |
| OIL | | |
| COCOS NUCIFERA | ACTIVATED | BIOTROPICS |
| (COCONUT) OIL | VIRGIN | |
| | COCONUT OIL | |
| TOCOPHEROL | DL ALPHA | DSM |
| | TOCOPHEROL | NUTRITIONAL |
| | | PRODUCTS |
| CETEARYL ALCOHOL | SINNOWAX ® AO | BASF |
| (and) CETEARETH-25 | | |
| POLYSORBATE 21 | SP TWEEN 21 | CRODA |
| | MBAL-LQ-(CQ) | |
| PHENOXYETHANOL | GLYSOLV EPHL | HUNTSMAN |
| | | (INDORAMA) |
| CHLORHEXIDINE | CHLORHEXIDINE | EVONIK |
| DIGLUCONATE | DIGLUCONATE | GOLDSCHMIDT |
| | SOLUTION/PURE | |
| | GRADE | |
| SIMMONDSIA | JD FFL JOJOBA | JOJOBA DESERT |
| CHINENSIS (JOJOBA) | GOLDEN OIL | |
| SEED OIL | | |
| ARGANIA SPINOSA | LIPOFRUCTYL | BASF BEAUTY |
| KERNEL OIL | ARGAN BE LS 9779 | CARE SOLUTION |
| PRUNUS ARMENIACA | APRICOT KERNEL | GUSTAV HEESS |
| (APRICOT) KERNEL OIL | OIL REFINED DAC | |
| Fluorescein sodium salt | LOT: MKCG7851 | SIGMA-ALDRICH |

### Example 1: Preparation of Compositions

Compositions according to comparative formula (Comp.) 1 and invention formulas (Inv.) 1-3 were prepared according to the contents given in Table 2 (the contents are expressed as weight percentages of active material relative to the total weight of each composition, unless otherwise indicated).

TABLE 2

| | Inv. 1 | Inv. 2 | Inv. 3 | Comp. 1 |
|---|---|---|---|---|
| Components | Wt. % | | | |
| C15-19 ALKANE | QS100 | QS100 | QS100 | — |
| CETEARYL ALCOHOL (and) CETEARETH-25 | — | — | — | 7.5 |
| SQUALANE | 10 | 10 | 10 | — |
| POLYSORBATE 21 | — | — | — | 4 |
| ETHYLHEXYL PALMITATE | 9 | 9 | 9 | — |
| PHENOXYETHANOL | — | — | — | 0.7 |
| POLY C10-30 ALKYL ACRYLATE | 3 | 1.5 | 0 | — |
| CHLORHEXIDINE DIGLUCONATE | — | — | — | 0.25 |
| HELIANTHUS ANNUUS (SUNFLOWER) SEED | 2.5 | 2.5 | 2.5 | — |
| OIL | | | | |
| SIMMONDSIA CHINENSIS (JOJOBA) SEED OIL | 2.5 | 2.5 | 2.5 | — |
| ARGANIA SPINOSA KERNEL OIL | 2.5 | 2.5 | 2.5 | — |
| PRUNUS ARMENIACA (APRICOT) KERNEL | 2.5 | 2.5 | 2.5 | — |
| OIL | | | | |
| WATER | — | — | — | QS100 |
| COCOS NUCIFERA (COCONUT) OIL | 0.9 | 0.9 | 0.9 | — |
| TOCOPHEROL | 0.5 | 0.5 | 0.5 | — |

Preparation Process:

The compositions listed above were prepared as follows, taking the composition of invention formula 1 as an example:
1). Mixing ETHYLHEXYL PALMITATE, HELIANTHUS ANNUUS (SUNFLOWER) SEED OIL, SIMMONDSIA CHINENSIS (JOJOBA) SEED OIL, ARGANIA SPINOSA KERNEL OIL, PRUNUS ARMENIACA (APRICOT) KERNEL OIL, COCOS NUCIFERA (COCONUT) OIL and POLY C10-30 ALKYL ACRYLATE to obtain a mixture and heating the mixture to 50° C. and homogenizing the mixture by stirring;
2). Adding C15-19 ALKANE and SQUALANE into the mixture and cooling down the temperature of the mixture to room temperature by stirring; and
3). Adding TOCOPHEROL into the mixture to obtain the composition.

### Example 2: Evaluation of Compositions

The appearance of each composition prepared in Example 1 was observed.

The viscosity, protection ability, and impact on dyeing and bleaching of each composition prepared in Example 1 were characterized.

Viscosity

The viscosity was measured at 25° C., using a Rheomat R180 viscometer equipped with a M3 or M2 spindle, the measurement being performed after 10 minutes of rotation of the spindle in the composition (after which time stabilization of the viscosity and of the spin speed of the spindle are observed), at a shear rate of 200 rpm.

The appearance and viscosity were summarized in Table 3.

TABLE 3

| Properties | Inv. 1 | Inv. 2 | Inv. 3 | Comp. 1 |
|---|---|---|---|---|
| Viscosity(UD) | 26.8(M3) | 30.5 (M2) | 8.9 (M2) | 25 (M3) |
| Appearance | Yellow | Yellow | Liquid | White |
| | balm | oil gel | yellow oil | cream |

Protection Ability

i) Evaluation by Confocal Microscopy

The in-vitro protect function of the compositions of invention formulas 2-3 and comparative formula 1 were evaluated with a confocal laser scanning microscope as follow:
1) Clean and dry a pig skin purchased from the market;
2) Apply the composition tested on the skin at 30 μl/cm²;
3) Prepare a dyeing composition as an irritation composition according to the contents given in Table 4 (the contents are expressed as weight percentages of active material relative to the total weight of each composition, unless otherwise indicated);

TABLE 4

| Components | Contents(wt. %) | Roles |
|---|---|---|
| AMMONIUM HYDROXIDE | 19.8 | Alkaline agents |
| ETHANOLAMINE | 0.63 | |
| oleth-30 | 3.6 | Surfactants (HBL) |
| CETEARYL ALCOHOL | 2.28 | Fatty compounds |
| EDTA | 0.2 | Chelating agents |
| Water | QS100 | solvent |
| Fluorescein sodium salt | 50 ppm | Water-soluble |
| | | fluorescent dye |

4) Apply the irritation sample on the skin at 75 μl/cm² for 40 minutes;
5) Wipe off with a tissue;
6) Choose the center area and frozen the section obtained;
7) Image with the microscope.

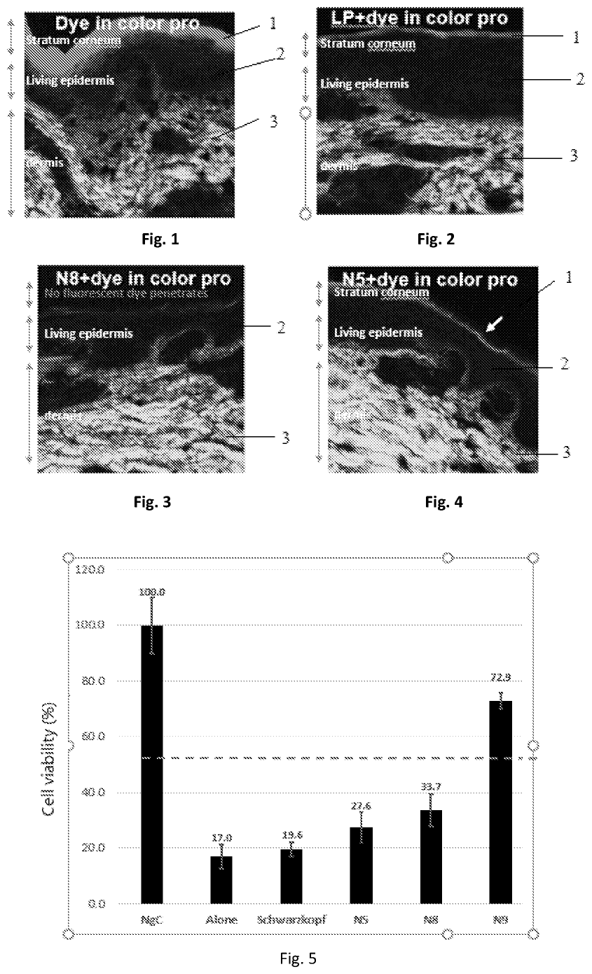

FIG. 1 shows the fluorescence intensity in stratum corneum upon application of a dyeing composition directly on the pig skin.

It can be seen from FIG. 1 that the dye in the dyeing composition can pass across the stratum corneum to the living epidermis, which may cause discomfort and irritation.

FIG. 2 shows the fluorescence intensity in stratum corneum upon application of the composition of comparative formula 1 and a dyeing composition.

FIG. 3 shows the fluorescence intensity in stratum corneum upon application of the composition of invention formula 2 and a dyeing composition.

FIG. 4 shows the fluorescence intensity in stratum corneum upon application of the composition of invention formula 3 and a dyeing composition.

It can be seen from FIGS. 2-4 that the compositions of invention formulas 2-3 can effectively block the dye penetrate into the skin, as compared with the composition of comparative formula 1. Meanwhile, the composition of invention formula 2 is more effective in terms of blocking the dye from penetrating into the skin.

ii) Evaluation by Episkin™

The in-vitro protection function of the compositions of invention formulas 1-3 and Pre-coloration protect oil from Schwarzkopf were evaluated by EpiSkin™ as follows.

Models:

EpiSkin™ reconstructed epidermis models were used.

EpiSkin™ tissues and medium were purchased from Shanghai EPISKIN Biotechnology Co., Ltd. The size of EpiSkin™ tissue is 1.07 cm².

For Bleach Products:

The composition tested (30 μl) were applied topically to the epidermal model (two epidermis units were used per test group). Then 75 μl of a mixture of a hair bleach powder (L'oreal professional blond studio 8 from the company L'oreal) and a developer (L'oreal professional oxidant cream from the company L'oreal) mixed at a volume ratio of 1:1.5 was applied topically to the epidermal model (45-minute-application at room temperature with a nylon mesh). Exposure was terminated by rinsing with phosphate buffered saline (PBS).

For Coloration Products

The composition tested (30 μl) were applied topically to the epidermal model (two epidermis units were used per test group). Then 75 μl of a mixture of a colorant (L'oreal professional Maji fashion colorant 12.11 without dye from the company L'oreal) and a developer (L'oreal professional oxidant cream from the company L'oreal) mixed at a volume ratio of 1:1.5 was applied topically to the epidermal model (45-minute-application at room temperature with a nylon mesh). Exposure was terminated by rinsing with phosphate buffered saline (PBS).

Post-incubation: Epidermis was then incubated at 37° C. for 18 hours.

The viability was assessed by incubating the tissues for 3 hours with a MTT solution. The precipitated formazan was then extracted using acidified isopropanol for 2 days at 4° C. and quantified spectrophotometrically at 570 nm using 96 well plates.

Epidermis without tested composition applied (bleach+coloration alone) and no treatment epidermis were used as positive and negative controls respectively.

FIG. 5 shows the amount of formazan for different treatments, wherein “Ngc” stands for the original cell viability, “alone” stands for the cell viability without protection, “shawarzkopf” stands for the cell viability under the protection of a product from Schwarzkopf, N5 stands for the cell viability under the protection of the composition of invention formula 3, N8 stands for the cell viability under the protection of the composition of invention formula 2, and N9 stands for the cell viability under the protection of the composition of invention formula 1.

It can be seen from FIG. 5 that the compositions of invention formulas 1-3 can effectively block chemicals such as ammonium hydroxide and ethanolamine from penetrating into the skin to irritate the scalp.

iii) Evaluation by Staining on a Via Skin

The in-vitro protect function of the compositions of invention formula 1 and comparative formula 1 were evaluated by staining on a pig skin.

FIG. 6 shows photos taken during evaluation by staining on a pig skin.

As illustrated in FIG. 6, the evaluation process includes:
1) Prepare a pig skin of 4 cm² and use a standard shampoo DOP (from L'oreal) wash the skin first, and then dry the skin;
2) Apply the composition tested on the pig skin at 30μ/cm²;
3) Apply a dyeing composition (a mixture of Maji fashion color NO. 1 from L'oreal and developer 30 from L'oreal mixed at a volume ratio of 1:2) at 75μ/cm² for 30 min;
4) Rinse off with DOP (the standard shampoo).

FIG. 6 shows photos taken during evaluation by staining on a pig skin, wherein “w/o protect” indicates no composition tested was applied, “w/scalp shield” indicates the compositions of invention formula 1 was applied, and “w/LP still cap” indicates the compositions of comparative formula 1 was applied.

It can be seen from FIG. 6 that the compositions of invention formula 1 can effectively block the dye from penetrating into the skin, as compared with the composition of comparative formula 1.

Impact on the Dyeing Effect of a Dyeing Product on the Hair

The impact of the compositions of invention formula 1 on the dyeing effect of a dyeing product on the hair was evaluated with hair swatches (Hair type: Chinese natural hair) and model's heads.

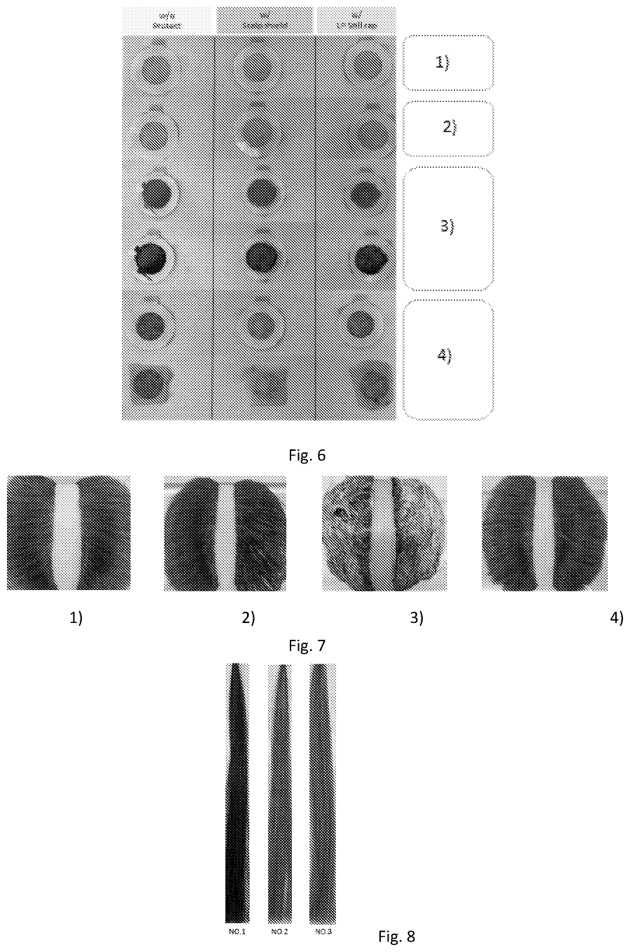

FIG. 7 shows photos taken during evaluation with hair swatches and model's heads.

As illustrated in FIG. 7, the evaluation process includes:
1) Wash the head 2 times with DOP (the standard shampoo, 12 g shampoo per full head for each test);
2) Apply 7.5 g of the composition tested on a half head and massage on the right half head for 1 minute;
3) Apply a dyeing product (150 g, a mixture of Maji 12.11 from L'oreal and Developer 30 from L'oreal mixed at a volume ratio of 1:2) on the full head;
4) Wash the full head with DOP for and observe the color differences between left and right.

It can be seen from FIG. 7 that there is no obvious color difference between the left head and the right head, indicating that the composition according to the present invention has no negative impact on the dyeing effect of a dyeing product on the hair.

Impact on the Bleaching Effect of a Bleaching Product on the Hair

The impact of the compositions of invention formula 1 on the bleaching effect of a bleaching product on the hair was evaluated with hair swatches (Hair type: Chinese natural hair).

FIG. 8 shows photos taken during evaluation with hair swatches.

As illustrated in FIG. 8, the evaluation process includes:
1) Select 3 hair swatches of the same (No. 1, No. 2 and No. 3) and keep hair swatch No. 1 untreated;
2) Apply the composition tested on hair swatch No. 2 first (0.15 g of the composition tested per gram of hair swatch No. 2), then apply a mixture of a hair bleach powder (L'oreal professional blond studio 8 from the company L'oreal) and a developer (L'oreal professional oxidant cream from the company L'oreal) mixed at a volume ratio of 1:1.5 on hair swatch No. 2 for 45 minutes (10 g of the mixture per gram of hair swatch No. 2);
3) Apply the same mixture of Bleach powder and Developer on hair swatch No. 3 for 45 minutes (10 g of the mixture per gram of hair swatch No. 3);
4) Compare the color of hair swatch No. 2 and hair swatch No. 3.

It can be seen from FIG. 8 that there is no obvious color difference between hair swatch No. 2 and hair swatch No. 3, indicating that the composition according to the present invention has no negative impact on the bleaching effect of a bleaching product on the hair.

1: An anhydrous composition for caring for the skin, comprising:
(i) at least one linear alkane containing from 10 to 28 carbon atoms;
(ii) at least one oil of formula R₁COOR₂ in which R₁ represents a linear or branched fatty acid residue containing from 4 to 40 carbon atoms and R₂ represents a hydrocarbon-based chain containing from 4 to 40 carbon atoms, on condition that R₁+R₂≥16; and
(iii) at least one vegetable oil of triglyceride type.

2: The composition according to claim 1, wherein the linear alkane is selected from linear C12-C24 alkane.

3: The composition according to claim 1, wherein the linear alkane is present in an amount ranging from 0.1 wt. % to 10 wt relative to the total weight of the composition.

4: The composition according to claim 1, wherein the oil of formula R₁COOR₂ is selected from esters of a C6-C24 monocarboxylic acid and of a C4-C20 alcohol, at least one of the acid and the alcohol being branched or unsaturated.

5: The composition according to claim 1, wherein the oil of formula R₁COOR₂ is selected from:
C4-C10 alkyl myristates;
C4-C10 alkyl palmitates;
C4-C10 alkyl isostearates, and
mixtures thereof.

6: The composition according to claim 1, wherein the oil of formula R₁COOR₂ is present in an amount ranging from 0.1 wt. % to 10 wt. %, relative to the total weight of the composition.

7: The composition according to claim 1, wherein the vegetable oils of triglyceride type are selected from the group consisting of sunflower seed oil, jojoba seed oil, Argania spinosa kernel oil, coconut oil, corn oil, soybean oil, marrow oil, grapeseed oil, sesame seed oil, hazelnut oil, apricot oil, macadamia oil, castor oil, and avocado oil.

8: The composition according to claim 1, wherein the vegetable oil of triglyceride type is present in an amount ranging from 1 wt. % to 30 wt. %, relative to the total weight of the composition.

9: The composition according to claim 1, further comprising a semi-crystalline or crystalline lipophilic thickener.

10: The composition according to claim 9, wherein the semi-crystalline or crystalline lipophilic thickener is selected from homopolymers of C8-C30 alkyl (meth)acrylate or C8-C30 alkyl(meth), copolymers of C8-C30 alkyl (meth)acrylate or C8-C30 alkyl(meth) and a hydrophilic monomer of different nature from (meth)acrylic acid, and mixtures thereof.

11: The composition according to claim 9, wherein the lipophilic thickener is present in an amount ranging from 0.1 wt. % to 10 wt. %, relative to the total weight of the composition.

12: The cosmetic composition according to claim 1 comprising, relative to the total weight of the composition:
(i) from 0.5 wt. % to 3 wt. % of at least one linear C15-C19 alkane;
(ii) from 0.5 wt. % to 3 wt. % of at least one C4-C10 alkyl palmitate;
(iii) from 8 wt. % to 15 wt. % of at least one vegetable oils of triglyceride type selected from the group consisting of sunflower seed oil, jojoba seed oil, Argania spinosa kernel oil, coconut oil, corn oil, soybean oil, marrow oil, grapeseed oil, sesame seed oil, hazelnut oil, apricot oil, macadamia oil, castor oil, and avocado oil; and
(iv) from 0.5 wt. % to 5 wt. % of at least one poly(C10-C30 alkyl acrylate).

13: A non-therapeutic method for caring for the skin, comprising applying the composition according to claim 1 to the skin.

14: The non-therapeutic method according to claim 13, wherein the composition is applied to the scalp.

15: The composition according to claim 1, wherein the linear alkane is selected from linear C14-C22 alkane.

16: The composition according to claim 1, wherein the oil of formula R₁COOR₂ is selected from esters of a C12-C20 monocarboxylic acid and of a C4-C10 alcohol, at least one of the acid and the alcohol being branched or unsaturated.
